(19) United States
(12) Patent Application Publication
(10) Pub. No.: US 2023/0000900 A1
(43) Pub. Date: Jan. 5, 2023

(54) METHOD FOR NEUTRALIZING STOMACH ACID USING ALGINATE, POLYLYSINE, AND SEED PRESERVATIVES

(71) Applicant: REFLUX GOURMET LLC, SACRAMENTO, CA (US)

(72) Inventor: Ken Frank, Napa, CA (US)

(21) Appl. No.: 17/856,542

(22) Filed: Jul. 1, 2022

Related U.S. Application Data
(60) Provisional application No. 63/218,488, filed on Jul. 5, 2021.

(51) Int. Cl. A61K 31/734; A61K 9/20; A61K 9/16; A61K 31/132; A61K 38/00; A61K 9/68

(52) U.S. Cl. CPC A61K 31/734; A61K 9/2054; A61K 9/1635; A61K 9/2059; A61K 31/132; A61K 38/005; A61K 9/0058

(57) ABSTRACT

Methods for neutralizing stomach acid in a subject including alginate-based nutritional products (“AP”). AP includes alginate as a reflux-neutralizing, raft-forming, stabilizing agent, including polylysine, dextrose and grapefruit seed extract preservatives. The methods are intended to provide relief or prophylaxis of acid reflux and other forms of indigestion in mammals. In some embodiments, dextrose and polylysine are combined to form a preservative for the alginate-based nutritional supplement. In other embodiments, a method for producing a reflux preventing raft is provided.

### PRIORITY

The application claims priority from the United States provisional application with Ser. No. 63/218,488, which was filed on Jul. 5, 2021. The disclosure of that provisional application is incorporated herein as if set out in full.

### TECHNICAL FIELD

The present disclosure relates generally to methods for neutralizing stomach acid in a subject using polylysine, dextrose and grapefruit seed extract as preservatives in an alginate-based nutritional supplement and digestive aid.

### BACKGROUND

Paraben-based compounds and popular synthetics are known in the art to act as effective preservatives and digestive aids. However, paraben-based compounds are known hormone disruptors and have been implicated in the development of certain cancers. Thus, there is a need in the art for safe, easy to manufacture, non-carcinogenic, non-hormone disrupting, non-paraben-based food preservatives and digestive aids. Alginates are natural polysaccharide polymers isolated from brown seaweed which may serve to partially address this need in the art. Alginates have been used safely as food additives for over one hundred years, with health benefits largely derived from their stable dietary fibers ease of digestion in the stomach of mammals.

The stomach may be considered an extension of the esophagus. Following an oral intake, food remains in the stomach between one and six hours. During that time, masticated food becomes more like a thin paste or gruel and is leaked in small portions to the duodenum. In order to process this food, several enzymes are produced by the epithelial cells. Furthermore, parietal cells produce hydrochloric acid, also called stomach acid, which is responsible for the low pH in the stomach. This stomach acid is a first-line immune response to foreign organisms, providing a sterilizing effect on bacteria, fungi and other microorganisms. Although essential to digesting food, stomach acid regurgitating back to the esophagus and upper airway is a problem in the modern world. The esophagus and throat (pharynx and larynx) are not equipped to handle the low pH stomach acid contents, and injury can occur. While many are genetically and physically predisposed to reflux, lifestyle factors such as stress, over-eating, eating or drinking too quickly, and gulping down carbonated drinks can further contribute to the regurgitation of stomach contents into the esophagus and upper airway.

Indigestion can be defined broadly as dyspepsia or GERD (Gastroesophageal reflux disease). Dyspepsia is a general term for the pain or discomfort a person can feel in the stomach and under the ribs, usually after eating or drinking (i.e., alcoholic beverages or coffee), although similar symptoms can occur on an empty stomach. GERD, on the other hand, is a digestive disorder caused by relaxation of the ring of muscle between one's esophagus and stomach. This ring is called the lower esophageal sphincter (LES), and the relaxation of it can further contribute to heartburn or acid indigestion.

Some people experience indigestion a few times per year while others suffer every day with symptoms ranging from mild discomfort lasting a few minutes to longer lasting severe pain, sometimes accompanied by nausea and vomiting, which can go on for several hours. The most common indigestion symptoms are: pain or discomfort in the stomach and under the ribs, heartburn, feeling of being bloated or uncomfortably full after eating, rumbling or gurgling noises in the stomach, stomach cramps, a clenched or knotted feeling in the stomach, excessive burping or flatulence, and nausea or vomiting. The most common symptoms of regurgitation of gastric contents into the upper airway, or laryngopharyngeal reflux (LPR), are throat pain, throat clearing, hoarseness, cough, and the sensation of a lump in the throat.

The disorder is experienced by an increasing number of people in the developed world. At least one third of the population suffers from episodic dyspepsia. Dyspepsia may be generally relieved by antacids, which work as bases or acid-buffering agents to raise the pH of the stomach contents. Antacids are normally over-the-counter products, which can be purchased at any pharmacy. Calcium carbonate is commonly used as the active ingredient in antacids. Some calcium-based antacids add other ingredients, such as magnesium or aluminum. Common over-the-counter antacids include Tums® sold by GlaxoSmithKline of Brentford, England, which is simply calcium carbonate; Rennie® sold by Bayer, which has magnesium added to ease the potential side effect of constipation with too much calcium; and Maalox®, sold by Novartis, which has aluminum added to calcium carbonate and comes as liquid or tablet forms. Antacids may be useful as self-medication in dyspeptic patients with mild or moderate heartburn, however, while they are fast-acting they are also short-acting, so they are less useful for frequent or severe heartburn and do not work well as a preventive measure.

Acid blockers such as H2 blockers and proton pump inhibitors (PPIs) are generally used for severe and chronic symptoms. These drugs work by blocking how much stomach acid is being produced. These acid blockers are not as fast-acting as antacids, but last longer and can be effective for several hours at a time. Over-the-counter acid blockers include Axid®, Pepcid®, Tagamet®, and Zantac®. These brands are also available in prescription strength if the milder forms do not bring enough relief. The clinical efficacy of H2 blockers and proton pump inhibitors is well documented (Hürlimann S, Michel K, Inauen W, Halter F: Effect of Rennie Liquid versus Maalox Liquid on intragastric pH in a double-blind, randomized, placebo-controlled, triple cross-over study in healthy volunteers. Am J. Gastroenterol. 1996; 91: 1173-1180; Feldman M: Pros and cons of over-the-counter availability of histamine2-receptor antagonists. Arch Intern Med 1993; 153; 2415-2424; Netzer P, Brabetz-Hoefliger A, Bruendler R, Flogerzi B, Huesler J, Halter F: Comparison of the effect of the antacid Rennie versus low dose; and H2-receptor antagonists (ranitidine, famotidine) on intragastric acidity. Aliment Pharmacol Ther 1998; 12: 337-342).

Acid blockers such as Pepcid®, Tagamet®, and Zantac®, work by blocking a type of histamine produced by the stomach, which in turn blocks acid production. These histamine blockers are typically taken twice a day, 30 to 60 minutes before eating to be most effective. Unfortunately, some histamine blockers have been implicated in the development of certain cancers, and many other serious, life threatening and/or life altering side effects.

Another class of acid blocking drugs called proton pump inhibitors, or PPIs, shut down tiny proton pumps in the stomach that produce acid, lowering acid levels dramatically. They are often used when histamine blockers do not provide enough relief or when people have erosions in the esophagus or other complications from GERD (Gastroesophageal reflux disease). One proton pump inhibitor, Prilosec®, is available over the counter in the US as well as numerous other countries. Other PPIs, such as Aciphex®, Nexium®, Prevacid®, Protonix®, and other forms of Prilosec® may require a doctor's prescription depending on the regional requirements. Reglan® is another prescription drug that works to reduce acid reflux by speeding up how quickly the stomach empties. Reglan® strengthens the digestive contractions that move food through one's esophagus, and with this faster digestion can be associated with reduced heartburn. Reglan® and other pro-kinetic drugs have potentially mortal side effects and received a “black box” warning from the Food and Drug Administration, the agency's strongest warning.

Alginates are all-natural derivatives of kelp (brown seaweed) that have been used safely as natural food additives for over a hundred years. Alginate is a common household ingredient that is used to thicken foods and make jellies. When consumed, alginates also coat the lining of the esophagus and stomach creating a protective demulcent coating and form a protective alginate raft that floats on top of stomach contents to provide relief against acid reflux when combined with bicarbonate in the stomach. One of the major challenges to developing natural alginate products to help provide reflux relief is that they can form a medium for bacterial and fungal growth and are very difficult to preserve. Because of this, the primary preservative used in contemporary alginate-based products is paraben. Paraben is a synthetic preservative, a known hormone disruptor that has been implicated in hair loss, allergic reactions, and in the development of numerous cancers.

In one example, British Patent No. 2324725 discloses a pharmaceutical composition suitable for forming a mucoadhesive lining in the gastrointestinal tract. It comprises an alginic acid or alginate salt with an M/G ratio of at least unity. The composition may be formulated as a liquid for treatment of reflux esophagitis. In the examples, 32 g calcium carbonate or 100 g of a 10% aluminum hydroxide gel is used per 100 g sodium alginate for the formation of a reflux preventing raft that floats on top of the stomach. However, the alginate product suffers from several adverse effects, including hormone disruption and potential carcinogenic effects from the paraben preservative required to prevent the growth of bacteria and fungi.

Relatedly, WO2001/087282A3 and US 2007/0281015 describe an antacid pharmaceutical composition for the rapid and prolonged neutralization of gastric acidity with mucosa-protecting activity. The pharmaceutical is intended as a liquid preparation for oral ingestion. It includes at least 30% of sodium alginate, an antacid soluble agent, and an inhibitor of proton pump, such as omeprazole. The antacid soluble agent of choice is sodium bicarbonate, which neutralizes hyperacidity acting directly in the digestive tract, the alginate forms a viscous suspension or gel after it has entered the stomach environment exerting protecting activity over gastric mucosa, and the inhibitor of a proton pump acts by selectively blocking the H+/K+-ATPase enzyme of stomach parietal cells.

It is the object of the present invention to disclose a nutritional aid/supplement (or “digestion aids”) containing alginates that are optimized for improved solubility, safety, taste and effectiveness. Said digestion aids benefit from nearly instantaneous action as well as longer lasting neutralizing effects on stomach acid, in contrast to the prior art described above. Thus, the herein described invention satisfies a need in art for safer, palatable, non-hormone disrupting, non-carcinogenic digestive aids, and in addition is all-natural, paraben-free, and safe.

### SUMMARY OF THE INVENTION

To minimize the limitations found in the existing systems and methods, and to minimize other limitations that will be apparent upon the reading of this specification, the present invention includes polylysine, dextrose, and grapefruit seed extract as preservatives in an alginate-based nutritional supplement product (“AP” or “digestive aid”).

The present disclosure provides methods for neutralizing stomach acid in a subject, including administering to a subject an effective amount of an alginate-based product (AP), wherein AP forms a reflux-preventing raft in the stomach of a subject, and wherein AP comprises alginate and polylysine. In some embodiments, AP is further adapted to neutralize acid reflux, and wherein AP further comprises a stabilizer, an alkaline salt, a secondary alkaline agent, and a salt comprising calcium.

In other embodiments, AP is adapted to neutralize reflux esophagitis, and wherein the alginate is formulated to inhibit proteolytic enzymes. In embodiments, inhibiting proteolytic enzymes includes inhibiting the proteolytic activity of pepsin and/or gastric juices. In some embodiments, AP is further adapted to neutralize dyspepsia, and wherein AP further comprises polylysine, dextrose, and grapefruit seed extract.

In some embodiments, AP is adapted to act both as an antacid and a reflux-preventing raft, wherein the reflux-preventing raft comprises a foam barrier adapted to neutralize stomach acid, acid reflux, reflux esophagitis, and/or dyspepsia by reacting with gastric acid and floating upon the contents of the stomach of the subject. In embodiments, AP forms a foam raft formulation with a 2.0 to 12.0% weight/volume of a low viscosity grade dextrose admixed a secondary preservative sugar.

In other embodiments, administering the effective amount of dextrose sugars contribute to desiccation of the product which facilitates its action as a preservative. Notably, AP may be formulated as a powder, syrup, and/or capsule. Also provided are methods for producing a reflux preventing raft. In addition, methods are provided for neutralizing reflux in a subject, including administering to a subject an effective amount of an alginate-based product (AP), wherein AP is administered in combination with at least a carrier and MTOR compound.

A first objective of the present invention is to provide methods for production and use of all-natural, safe, non-hormone disruptive, non-carcinogenic digestion aids.

A second objective of the present invention is to provide methods of use of a digestion aid or alginate product (“AP”) that is more palatable for the user, coming in a variety of unique natural flavors optimized by a world class chef.

It is another objective of the present invention to optimize alginate raft technologies such that lower concentrations of active alginate ingredients are required for a therapeutic effect.

Yet another object of the invention is to optimize the manufacturing of the indigestion related nutritional supplement/digestive aid claimed herein, providing improved solubility for several ingredients including salts.

These and other advantages and features of the present invention are described with specificity so as to make the present invention understandable to one of ordinary skill in the art. The following detailed description together with accompanying figures will provide a better understanding of the nature and advantages of the present invention.

### BRIEF DESCRIPTION OF THE DRAWINGS

In order to enhance clarity and improve understanding of the various elements and embodiments of the invention, elements in the figures have not necessarily been drawn to scale. Furthermore, elements that are known to be common and well understood to those in the industry are not depicted in order to provide a clear view of the various embodiments of the invention. Thus, the drawings are generalized in form in the interest of clarity and concision.

FIG. 1 shows the chemical structure for polylysine in accordance with the preferred embodiment of the present invention;

FIG. 2 shows the chemical structure for dextrose in accordance with the preferred embodiment of the present invention;

FIG. 3 shows the chemical structure for alginic acid (“alginates”) in accordance with the preferred embodiment of the present invention; and

FIG. 4 shows a diagrammatic image of an alginate raft precipitate.

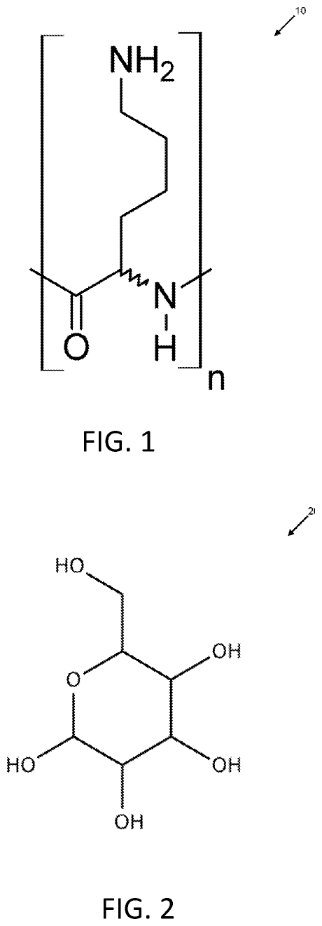

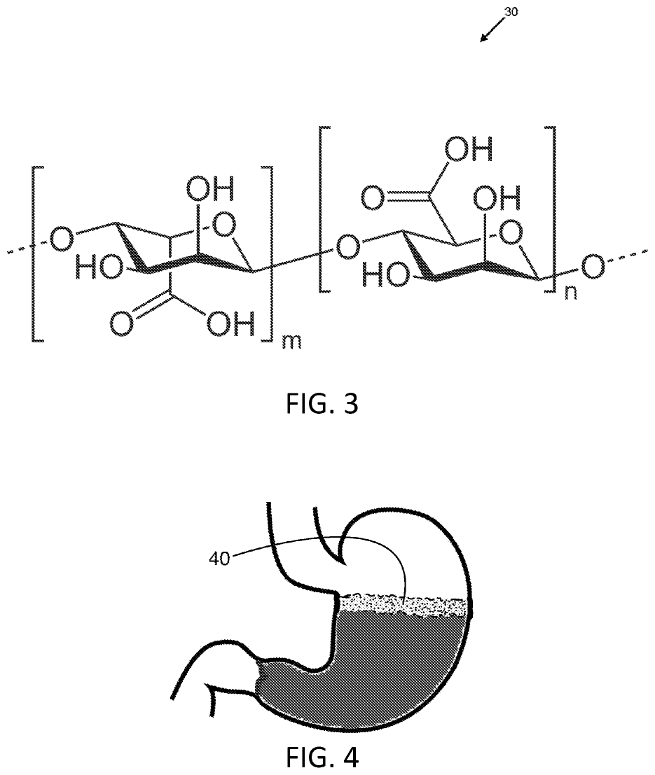

### DETAILED DESCRIPTION OF THE INVENTION

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms “a”, “an”, and “the” include plural referents unless the context clearly dictates otherwise. “And” as used herein is interchangeably used with “or” unless expressly stated otherwise. As used herein, the term “about” means +/−5% of the recited parameter. All embodiments of any aspect of the invention can be used in combination unless the context clearly dictates otherwise.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In some embodiments, the use of polylysine 10, dextrose 20 and grapefruit seed extract as preservatives in an alginate-based product is contemplated. In the preferred embodiment, the alginate-based product (hereafter referred to as “AP” or “alginate supplement”) is a composition that includes an alginate-based product using alginate 30 as a stabilizing agent, in addition to polylysine 10, dextrose 20 and grapefruit seed extract preservatives. In some embodiments, dextrose 20 (or other common sugars known in the art) and polylysine 10 are combined to form a preservative for the alginate-based product. In other embodiments, dextrose 20 (or other common sugars known in the art), grapefruit seed extract, and polylysine 10 are combined to form a preservative for the alginate-based product.

In some embodiments, the dextrose 20 sugars, and/or other sugars, contribute to desiccation of the product which facilitates its action as a preservative. In addition, in some embodiments AP forms a demulcent (protective) barrier on the mucosal lining of the throat and esophagus of a user in order to provide relief against the partially regurgitated low pH constituents of the small intestine and stomach characteristic of gastroesophageal reflux. Once in the stomach, and as shown in FIG. 4. AP forms a stable protective raft 40 of foam-like composition (also referred to herein as a “gel raft”, “foam”, or “foam raft”) that serves as a pH neutral mechanical barrier reducing gastric regurgitation from the stomach to the esophagus and throat.

Reduction in regurgitation reduces exposure of the throat of a user to the low pH contents of the stomach, which otherwise contributes to local inflammation, DNA damage, pain, halitosis, and poor sleep.

In the preferred embodiment, AP comprises 200 mg sodium alginate, 170 mg Vitamin B5, 200 mg sodium bicarbonate, dextrose 20, polylysine 10, and grapefruit seed extract. In the preferred embodiment, the alginate gel coats the tissues of the throat and esophagus, providing relief against acid reflux and other forms of indigestion described above. Notably, the 200 mg Sodium Alginate is used as a food additive, forming a foam-like and/or viscous solution or gel. In addition, the 170 mg Vitamin B5 (calcium pantothenate) enhances the strength and cohesiveness of the foam-like raft. Calcium increases raft strength by cross-linking alginic acid polymers 30. In some embodiments, Vitamin B5 provides the unexpected result of enhanced raft strength and reduced acid reflux. Further, Vitamin B5 helps the body convert masticated carbohydrates into glucose, which the body uses to produce Adenosine Triphosphate (ATP) via aerobic metabolism. These B vitamins, often referred to as B complex vitamins, also help the body metabolize fats and protein and contribute to improved nervous system function. The AP further serves to fill the stomach and can be used as a diet aid.

Further to the above, 200 mg Sodium Bicarbonate enhances the utility and potency of the raft formation. Notably, in the presence of gastric acid, the bicarbonate forms carbon dioxide bubbles within the alginate gel matrix, in the process, buffering out acid. This feature induces foaming action in the raft and further allows the gel to float on the surface of the gastric contents. Carbon dioxide also contributes to the acid neutralizing effect of AP. In some embodiments, dextrose 20 serves as an all-natural sweetener and essential preservative. As described above, dextrose 20 also reduces the water content of AP which is essential to the preservative function of AP. The unique combination of alginate 30 and dextrose 20 significantly enhances the acid reflux-dampening effect of AP in a manner not contemplated by the art. As described above, polylysine 10 also serves as an essential all-natural preservative. In some embodiments, grapefruit seed extract (“GSE extract”) serves as an essential all-natural preservative, antifungal, and sweetener providing a unique high concentration of antioxidants.

In some embodiments, AP is a low sodium, iodine-free, potassium-free alternative to many anti-reflux compositions known in the art. Other products known in the art contain potential carcinogens such as paraben, a known hormone disruptor, as well as high salt content, high concentrations of potassium, contributing to high blood pressure and kidney injury in some patients.

In contrast, all ingredients of AP are non-synthetic, thus ameliorating digestive and organ filtering issues. In another embodiment, AP serves as a dieting aid with the utility of AP largely derived from its fibrous compounds. In the stomach of a user, said compounds expand, making the user feel satiated while contributing to a well-balanced pH environment.

Expanding on the utility of Vitamin B5, aside from its digestive functions, the compound is highly water soluble and is critical to the manufacture of red blood cells. Further, Vitamin B5 acts as a substrate for the synthesis of stress-related hormones produced in the adrenal glands. In some embodiments, Vitamin B5 in AP facilitates maintenance of a healthy digestive tract and helps the body use other vitamins, particularly B2 (also called riboflavin). Notably, the miscibility of calcium and Vitamin B5 were contemplated by the present inventors. In some embodiments, the ideal composition of ingredients (optimized for solubility) included a Chilean seaweed sourcing of alginate 30 (specifically, Lessonia nigrescens) formulated at a ratio of 60/40 (M/G) ratio.

In some embodiments, various dosages of each of the above ingredients are contemplated, with each optimized for buffering, antioxidant capacity, and the like, tailored to each particular foodstuff. As described above, in the preferred embodiment AP comprises 200 mg sodium alginate, 170 mg Vitamin B5, 200 mg sodium bicarbonate, dextrose 20, polylysine 10, and grapefruit seed extract. In another embodiment, AP comprises a range of 75-400 mg sodium alginate, 100-350 mg Vitamin B5, 100-300 mg sodium bicarbonate, with dextrose 20, polylysine 10, and grapefruit seed extract included at non-specific dosages.

In some embodiments, AP relates to the relief of reflux esophagitis using alginates 30 specifically formulated to inhibit proteolytic enzymes. In some embodiments, the invention relates to inhibiting the proteolytic activity of pepsin and/or gastric juices. In other embodiments, one or more dextrose molecules 20 with a preferred molecular weight of less than 500 kDa are used in combination with polylysine 10, dextrose 20 and grapefruit seed extract preservatives. The composition may be in the form of a dry powder, which can be admixed with water. Furthermore, the preparations may be composed in liquid form preferably containing the amount of from 0.1 to 15% w/v of dextrose 20. In some embodiments, the composition further comprises a neutralizing agent for neutralizing gastric acid such as sodium hydroxide.

In some embodiments, the composition preferably comprises divalent or trivalent metal cations to strengthen the formation of the foamy raft. The cations may be calcium or aluminum ions, for example. For instance, the composition may contain from 10 to 80 g of calcium carbonate per 100 g dextrose 20. In some embodiments, AP exercises its effect by dissolving solid calcium carbonate salt in the stomach under the influence of the acid gastric fluid, in addition to polylysine 10, dextrose 20 and grapefruit seed extract. The increasing calcium concentration will stimulate the alginate gelation as calcium ions and the polysaccharides form a rigid matrix. Furthermore, the dissolving of calcium carbonate and sodium bicarbonate liberates CO₂ gas which will be entrapped in the alginate matrix thereby forming a foam raft. Apart from the potential carcinogens and known hormone disruptors in the art in addition to the inconvenience of the consumption an inhomogeneous product, products known in the prior art have the disadvantage that the consumer may not obtain the calcium salt in a proper dose for an optimal gelation to progress. More importantly, the sodium alginate salts used in the prior art are not available to react with the acid present in the stomach because it is occupied with calcium ions in a cage-like structure.

To remedy this problem, in some embodiments AP can act as both an antacid and a foam barrier or raft where the esophagus empties into the top of the stomach. When the tablet is swallowed or the liquid is ingested, the antacid, polylysine 10, dextrose 20 and grapefruit seed extract neutralize stomach acid and the foaming agent creates a physical barrier that helps provide relief from acid reflux. The compositions may be administered orally in the form of a dry powder or aqueous suspension which may also contain a suspending agent and/or a preservative. The antacid/foam formulation reacts with gastric acid to form a raft on the contents of the stomach. In some embodiments, AP comprises a foam formulation with a 2.0 to 12.0% weight/volume of a low viscosity grade dextrose admixed a secondary preservative sugar. Relatedly, a liquid formulation of the antacid/foam formulation comprises an aqueous medium containing 1.5 to 10.0% weight/volume of a low viscosity grade dextrose 20 admixed with other sugars known in the art. To suspend the calcium carbonate particles in the aqueous medium a suspending agent like acrylic polymer cross-linked with 1% dextrose 20 may be used.

In other embodiments, improved pourability is obtained in an AP formulation wherein a liquid composition comprising potassium bicarbonate instead of other bicarbonates and at least 10% w/v sodium alginate is used in combination with polylysine 10, dextrose 20 and grapefruit seed extract as preservatives. The composition obtains a viscosity which does not possess thickening problems even when stored at low temperatures. Salt of divalent metal ions, such as calcium carbonate, are generally included in the compositions described above in an amount of 5 to 40 g/100 g dextrose 20 in order to obtain an optimal foam-like raft formation.

In some embodiments, a method for providing relief from reflux esophagitis as well as dyspepsia using all natural polylysine 10, dextrose 20 and grapefruit seed extract as preservatives is contemplated. As described above, this method further relates to the preparation of a pourable liquid sodium alginate composition. The composition preferably comprises a dextrose 20 content totaling 8 to 15% w/v. In this example, the composition further comprises an amount of bicarbonate. Bicarbonate may be required in order to produce adequate carbon dioxide in the stomach to obtain a proper raft formation 20.

In some embodiments, the composition of the invention may contain a minor amount of an alkaline salt comprising a cross-linking polyvalent metal ion to support the foam-like raft, if desired, when contacted with the gastric juice. In some embodiments, the raft is mechanically acting, meaning its reflux-preventing actions stem from physically obscuring access to substrate. Notably, the cross-linking polyvalent metal ion may be ions of calcium and aluminum and the salt may as an example be selected among calcium carbonate, CaHPO₄, aluminum carbonate and aluminum hydroxide. The salt comprising the cross-linking polyvalent metal ion is usually present in an amount of less than 12% weight, such as less than 5% by weights, such as less than 1% by weights, such as less than 0.3% by weights, preferably less than 0.1% weight, based on the weight of the alginate. The salt is generally solid in the composition but is suitable highly dissolvable at a pH present in the stomach. In an aspect of the invention the salt provides less than 200 ppm polyvalent metal ions when the pH of the composition is changed to about pH=2. In one aspect the amount of calcium salt in the composition provides less than 50 ppm dissolved calcium ions when the pH is changed to about pH=2.

In some embodiments, AP further comprises an alkaline salt, wherein the alkaline salt is a disintegrating agent selected from the group consisting of sodium bicarbonate (NaHCO₃), potassium bicarbonate (KHCO3), and mixtures thereof. In some embodiments, AP further comprises at least 2%, 4%, 8%, 10% by weight or more, based on the weight of the sodium alginate, of a further alkaline agent selected among the group consisting of magnesium hydroxide (Mg(OH)2), potassium hydroxide (KOH), sodium hydroxide (NaOH), sodium acetate (C2H3O2Na), and mixtures thereof. In some embodiments, AP further comprises less than 10%, 8%, 6%, or 4% by weight based on the weight of the sodium alginate of a salt comprising calcium. In some embodiments, the composition further comprises an alkaline agent such as magnesium hydroxide. In some embodiments, the composition further comprises an amount of sodium bicarbonate totaling 10%, 12%, 15%, or 20% by weight or more, based on the weight of the sodium alginate. In other embodiments, the amount of magnesium hydroxide is 1%, 2%, or 3% by weight or more, based on the weight of the sodium alginate.

As described above, in the preferred embodiment the alginate-based product (“AP”) is a composition that includes alginate 30 as a stabilizing agent, in addition to polylysine 10, dextrose 20, grapefruit seed extract preservatives, with an optional minor amount of salt. In other aspects, however, the composition of the invention (“AP”) does not contain an added salt comprising a cross-linking polyvalent metal ion, such as a calcium salt, said salt being capable of dissolving at a pH present in the stomach. Insignificant amounts of calcium salts may be present in, for example, the tap water ingested by a user of the composition. In one embodiment, at least 70%, such as 80%, preferably at least 90% of the alkali metal carboxylic acid groups of the alkali metal alginate are available for reaction with the stomach acid, i.e., is not reacted with a cross-linking polyvalent metal ion. In some embodiments, the agent comprising calcium is Vitamin B5 and/or another B complex vitamin known in the art. Calcium pantothenate is alternately known as pantothenic acid or vitamin B5 or panthenol, which is a composition of 8% calcium and 92% pantothenic acid.

The compositions according to the present invention may comprise one or more acceptable carriers in addition to the active constituent(s) described above. The carrier(s) must be “acceptable” in the sense of being compatible with the other ingredients of the composition and not deleterious to the recipients thereof. In a certain embodiment, the alginate is purified. The composition may be formulated as tablets, pills, syrups, capsules, gummies, powders, etc.

It will be appreciated that the amount of the composition required for treatment or prevention will vary according to the form of administration, the disorder to be addressed, the condition, age, the history of the subject, and the formulation of the composition, etc. When a person is seeking relief from a certain affliction, the amount of active components are preferably effective to provide relief for the individual. Often with increasing age, an increased dosage of AP is required to impart the palliative effects of the GSE antioxidants.

The term “carrier” refers to a diluent, adjuvant, excipient, or vehicle with which the active components are administered. The carriers in the composition may comprise a binder, such as microcrystalline cellulose, polyvinylpyrrolidone (polyvidone or povidone), gum tragacanth, gelatin, starch, lactose or lactose monohydrate; a lubricant or surfactant, such as magnesium stearate, or sodium lauryl sulphate; a glidant, such as colloidal silicon dioxide; a sweetening agent, such as dextrose 20, sucrose or saccharin; and/or a flavoring agent, such as peppermint, methyl salicylate, orange flavoring, and/or other natural flavors.

In some embodiments, the AP formulation further comprises one or more enzyme cofactors, and/or one or more essential nutrients. Exemplary cofactors include vitamin C, biotin, vitamin B5, vitamin E, and vitamin K. Exemplary essential nutrients are amino acids, fatty acids, etc. AP may also comprise one or more excipients known in the art, such as dispersing agents, wetting agents, and suspending agents. Solutions, suspensions, or emulsions of AP may also contain one or more excipients known in the art, such as dispersing agents, wetting agents, and suspending agents.

Therapeutic formulations suitable for oral administration, e.g., tablets and pills, may be obtained by compression or molding, optionally with one or more accessory ingredients. Compressed tablets may be prepared by mixing the constituent(s) and compressing this mixture in a suitable apparatus into tablets having a suitable size. In one embodiment, prior to the mixing, an alkaline metal alginate may be mixed with a binder, a lubricant, an inert diluent and/or a disintegrating agent and the alkaline salt may be mixed with a diluent, a lubricant and/or a surfactant.

In some embodiments, when one or more carriers are present, free-flowing alkaline metal alginate powder may be mixed with a binder, such as microcrystalline cellulose, and a surfactant, such as sodium lauryl sulphate, until a homogeneous mixture is obtained. Subsequently, another binder, such as polyvidone, may be transferred to the mixture under stirring. When a uniform distribution is obtained the alkaline salt is added under constant stirring. This mixture is passed through granulating sieves and dried by desiccation before compression into tablets in a standard compressing apparatus. Alternatively, the tablets are prepared without the addition of carriers. According to this alternative embodiment the free-flowing alkaline metal alginate is mixed with an alkaline salt before the mixture is compressed into tablets. Conventional methods for producing a solid form of the composition may be found in the European Pharmacopoeia, which is included herein by reference. A tablet may be coated or uncoated. An uncoated tablet may be scored. A coated tablet may be coated with sugar, shellac, film or other enteric coating agents.

In other embodiments, grape seed compounds comprise natural seed antioxidants, fibers, and MTOR compounds (e.g., compounds shown to act on MTOR gene signaling pathways including resveratrol). In other embodiments, inclusion and concentration of resveratrol enhances both the potent antioxidant features of AP but may also contribute neuro-protective and life-extension benefits to user. Notably, resveratrol is highly concentrated in grape skins and has been shown to extend the life span of certain laboratory mice of up to 50% when administered in high concentrations. This observation is likely linked both to the potent antioxidant action of resveratrol along with a gene-signaling/gene-regulatory effects acting through the MTOR signaling pathways, for example.

In some embodiments, there are multiple flavors of AP including vanilla caramel, mint chocolate, and/or other natural flavors. These flavors derive in part from natural antioxidant components and sugars inherent to the natural ingredients. In the preferred embodiment, AP also has a basic pH and/or infuses pH buffering compounds such that acid reflux is lessened for users. In this manner, AP can be taken as both a flavorful foodstuff and a supplement for acid reflux by a user. One of the unique advantages of AP is the highly flavorful product (intensity of flavor), in addition to the variety of flavors (wide range of flavors).

In some embodiments, the reflux preventing raft (“raft”) is produced by the steps: a) providing a functionalized natural or synthetic calcium carbonate comprising mineral, wherein said functionalized natural or synthetic calcium carbonate is a reaction product of natural or synthetic calcium carbonate with carbon dioxide and one or more acids, wherein the carbon dioxide is formed in situ by the acid treatment and/or is supplied from an external source; b) providing at least one polylysine-containing ingredient; c) providing at least one formulating agent comprising alginate; d) mixing the compounds provided in steps a) b) and c); e) granulating the mixture obtained in step d) by way of melt, dry or wet granulation or roller compaction. Notably, step “c” may include the addition of dextrose, preservatives, grapefruit seed extract, and other secondary compounds.

Further to the above, in some embodiments, parts of the formulation aid of step c) is first mixed with the functionalized calcium carbonate of step a) and the at least one polylysine-containing ingredient of step b), and the remaining portion of the formulation aid is then added to the mixture, followed by the granulation step e). Further to the above, in some embodiments, the source of natural calcium carbonate for preparing the functionalized calcium carbonate is selected from the group of marble, calcite, chalk, Vitamin B5, limestone and dolomite and/or mixtures thereof. In other embodiments, the synthetic calcium carbonate for preparing the functionalized calcium carbonate is precipitated calcium carbonate comprising aragonitic, vateritic or calcitic mineralogical crystals forms, especially prismatic, rhombohedral or scalenohedral precipitated calcium carbonate or mixtures thereof. In still other embodiments, the acids are selected from the group of hydrochloric acid, sulfuric acid, sulfurous acid, hydrosulfate, phosphoric acid, phosphoric acid in combination with acetic, formic or citric acid or acid salts thereof, and mixtures thereof, preferably is phosphoric acid.

In some embodiments, the functionalized natural or synthetic calcium carbonate has a specific surface area of from 5 m²/g to 200 m²/g, preferably 20 m²/g to 150 m²/g, more preferably 40 m²/g to 100 m²/g, measured using nitrogen and other methods known in the art. In some embodiments, the functionalized natural or synthetic calcium carbonate has a weight median grain diameter of from 0.2 to 40 μm, preferably from 0.6 to 25 μm, more preferably from 0.9 to 18 μm, still more preferably from 1 to 15 μm, measured using standard means known in the art such as Malvern Mastersizer X long bed.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. A method for neutralizing stomach acid in a subject, comprising: administering to a subject an effective amount of an alginate-based product (AP), wherein AP forms a raft in the stomach of the subject, and wherein AP comprises alginate and polylysine.

2. The method of claim 1, wherein AP and the raft are adapted to neutralize acid reflux, and wherein AP comprises a stabilizer, an alkaline salt, a secondary alkaline agent, and a salt comprising calcium, wherein a raft floats on top of stomach contents.

3. The method of claim 2, wherein AP is further adapted to neutralize reflux esophagitis, and wherein the alginate is formulated to inhibit proteolytic enzymes.

4. The method of claim 3, wherein inhibiting proteolytic enzymes comprises inhibiting the proteolytic activity of pepsin and/or gastric juices.

5. The method of claim 4, wherein AP is further adapted to neutralize dyspepsia, and wherein AP further comprises polylysine, dextrose, and grapefruit seed extract.

6. The method of claim 5, wherein AP is adapted to act both as an antacid and as a mechanically acting, reflux-preventing raft, wherein the reflux-preventing raft comprises a foam barrier adapted to neutralize stomach acid, acid reflux, reflux esophagitis, and/or dyspepsia by reacting with gastric acid and floating on top of the contents of the stomach.

7. The method of claim 5, wherein AP comprises a foam formulation with a 2.0 to 12.0% weight/volume of a low viscosity grade dextrose admixed with a secondary preservative sugar.

8. The method of claim 5, wherein administering the effective amount of dextrose sugars contributes to desiccation of the product which facilitates its action as a preservative.

9. The method of 5, wherein AP is formulated as a powder, syrup, capsule, gel and/or gummy.

10. The method of claim 9, wherein AP is administered in combination with one or more cofactors, excipients, vitamins, carriers, surfactants, and/or binders.

11. A method for producing a reflux preventing raft comprising the steps:
a) providing a functionalized natural or synthetic calcium carbonate comprising mineral, wherein said functionalized natural or synthetic calcium carbonate is a reaction product of natural or synthetic calcium carbonate with carbon dioxide and one or more acids, wherein the carbon dioxide is formed in situ by the acid treatment and/or is supplied from an external source;
b) providing at least one polylysine-containing ingredient;
c) providing at least one formulation agent, the formulation agent comprising alginate, dextrose, and/or grapefruit seed extract;
d) mixing the compounds provided in steps a) b) and c); and
e) granulating the mixture obtained in step d) by way of melt, dry, or wet granulation or roller compaction.

12. The method of claim 11, wherein the parts of the formulation agent of step c) are first mixed with the functionalized calcium carbonate of step a) and the at least one polylysine-containing ingredient of step b), and the remaining portion of the formulation agent is then added to the mixture, followed by the granulation step e).

13. The method of claim 12, wherein the source of natural calcium carbonate for preparing the functionalized calcium carbonate is selected from the group of marble, calcite, chalk, Vitamin B5, limestone and dolomite and/or mixtures thereof.

14. The method of claim 11, wherein the synthetic calcium carbonate for preparing the functionalized calcium carbonate is precipitated calcium carbonate comprising aragonitic, vateritic or calcitic mineralogical crystals forms, especially prismatic, rhombohedral or scalenohedral precipitated calcium carbonate or mixtures thereof.

15. The method 14, wherein the acids are selected from the group of hydrochloric acid, sulfuric acid, sulfurous acid, hydrosulfate, phosphoric acid, phosphoric acid in combination with acetic, formic or citric acid or acid salts thereof, and mixtures thereof, preferably is phosphoric acid.

16. The method of claim 15, wherein the functionalized natural or synthetic calcium carbonate has a specific surface area of from 5 m²/g to 200 m²/g, preferably 20 m²/g to 150 m²/g, more preferably 40 m²/g to 100 m²/g.

17. The method of claim 15, wherein the functionalized natural or synthetic calcium carbonate has a weight median grain diameter of from 0.2 to 40 μm.

18. A method for neutralizing acid reflux in a subject, comprising: administering to a subject an effective amount of an alginate-based product (AP), wherein AP is administered in combination with at least a carrier, grapefruit seed extract, and an MTOR compound.

19. The method of claim 18, wherein the MTOR compound comprises resveratrol and wherein resveratrol enhances both the antioxidant features of AP and the neuro-protective benefits of AP for the subject.

20. The method of claim 19, wherein the carrier comprises microcrystalline cellulose, polyvinylpyrrolidone, gelatin, starch, and/or lactose; and wherein AP further comprises a lubricant or surfactant, the surfactant comprising magnesium stearate and/or sodium lauryl sulphate.
